(19) United States
(12) Patent Application Publication
(10) Pub. No.: US 2016/0058695 A1
(43) Pub. Date: Mar. 3, 2016

(54) Topical Composition and Method for Skin Rejuvenation

(71) Applicant: Hunter Reid Moyer, Cumming, GA (US)

(72) Inventor: Hunter Reid Moyer, Cumming, GA (US)

(21) Appl. No.: 14/472,411

(22) Filed: Aug. 29, 2014

(51) Int. Cl. A61K 8/98; A61K 8/64; A61Q 19/08

(52) U.S. Cl. CPC A61K 8/981; A61Q 19/08; A61K 8/64; A61K 2800/70

(57) ABSTRACT

A skin rejuvenation process combining skin resurfacing with post-procedure application of a composition including the patient's own mesenchymal hematopoietic stern cells and growth factors is described. The stem cells and growth factors are isolated from lipoaspirate or bone marrow. In certain embodiments, the skin may be resurfaced by chemical, light or mechanical means to remove the main diffusion barrier, the stratum corneum. In additional embodiments, the stem cells and growth factors are minimally processed and applied to the skin in the form of a topical serum, cream, emollient or lotion.

### BACKGROUND OF THE INVENTION

1. Field of the Invention

Embodiments of the present invention are directed to a composition containing autologous stem cells and growth factors and a process to prepare the skin for topical application and penetration of the composition to ameliorate irregularities of the skin.

2. Background of the Art

Human skin is exposed to a multitude of environmental stressors throughout daily life. In addition, the inherent production of collagen, elastin and melanin decreases as we age. Accordingly, as humans age the presence of wrinkles and skin irregularities increase.

Known methods to smooth wrinkles, correct skin irregularities and replace volume include resurfacing procedures, botulinum toxin injection, and the injection of subcutaneous and/or dermal fillers. Results from these procedures are temporary and require the body's natural healing response to repair the tissue injury that occurs. The body's ability to heal determines the time delay to see results, efficacy of the procedure, and the duration of skin improvement.

Current techniques for skin resurfacing incorporate a myriad of creams and bases to help the healing process. One such method includes the application of the patients own platelet rich plasma (“PRP”) to the healing skin. As studies and laboratory investigations have proven, the release of growth factors from the alpha granules of platelets last a maximum of 24 hours after which minimal growth factors are produced. Therefore, a single application of PRP has a very limited time to affect change or patients have to have multiple, serial blood draws to treat healing skin. In addition, platelets hold a limited type of growth factors (TGFbeta, PDGF and ILGF1).

### BRIEF SUMMARY OF THE INVENTION

Embodiment of the present invention include a cosmetic composition comprising somatic stem cells and growth factors derived from bone marrow or adipose tissue. The stem cells and growth factors may be included in a serum, lotion, emollient, or cream. Still further the cosmetic composition may further comprise at least one amino acid selected from the group consisting of phenylalanine, valine, threonine, tryptophan, methionine, leucine, isoleucine, lysine, histidine, cystine, tyrosine, and glutamine.

Embodiments of the present invention also include a method for treating skin of a patient comprising the steps of isolating somatic stem cells and growth factors from the patient; preparing a cosmetic composition comprising the somatic stem cells and growth factors; disrupting the epidermis diffusion barrier of the skin to be treated to form a disrupted epidermis diffusion barrier skin region; and applying the cosmetic composition to the disrupted epidermis diffusion barrier skin region.

The method may include isolating the somatic stem cells and growth factors from using bone marrow or adipose tissue. Still further, embodiments of the invention may includ disrupting the epidermis diffusion barrier of the skin is selected from the group consisting of chemical peel, laser, mechanical abrasion, needling and rolling. Preferably, the cosmetic composition to a patient is performed within 48 hours after the step of preparing the cosmetic composition and more preferably within 5 hours after the step of preparing the cosmetic composition. Advantageously, in some embodiments the cosmetic composition is prepared from the patient's lipoaspirate or blood.

### DETAILED DESCRIPTION OF PREFERRED EMBODIMENTS OF THE INVENTION

Autologous stem cells are a durable source of self-renewing cells that can remain healthy and secrete a wide range of growth factors over a long period of time. Stem cells are innate to all animal and human tissues. They have the ability to self-renew and display the ability to differentiate into a range of cell types. Adult stem cells are termed somatic stem cells, as they are not derived from embryos. Most somatic stem cells are lineage restricted (multipotent, instead of the pluripotency or totipotency of embryonic stem cells) and are generally referred to by their tissue origin—hematopoietic stem cell, mesenchymal stem cell, etc.

Stem cells renew and generate new tissue both by mitotic division of the cell as well as paracrine signaling to neighbor cells—both stem cells and differentiated cells. In areas injected with stem cells, new dermal collagen formation, enhanced epithelialization, and improved wound healing has been observed. The role of paracrine signaling from blood and circulating stem cells in healing, and a body of research has focused on both natural homing of and exogenous delivery of stem cells to injured tissues.

Signs of skin aging (rhytids, poor elasticity, heterochromia, etc.) result from cellular and extracellular matrix phenomenon in the papillary and reticular dermis. Therefore, for any topical cosmetic—cellular or non-cellular—to affect change, the products must penetrate through the outer epidermis into the dermis or be injected underneath the skin.

Often techniques to deliver stem cells to the dermis are limited to injection techniques. An effective way to topically deliver stem cells and/or signaling molecules is lacking. The stratum corneum layer of the epidermis is specifically designed to limit water loss and prevent the penetration of environmental toxins. As such, a topical agent must be lipophilic and small in weight (generally less than 600 Daltons) to absorb into the dermis. Most proteins, all types of collagen, and all cells are much larger than 600 Daltons, and thus do not penetrate into the deeper layers of skin.

The present invention addresses this need and embodiments of the invention may include a method in which (i) a cosmetic preparation is prepared from the patient's stem cells and growth factors isolated from bone marrow or adipose tissue, (ii) the epidermis diffusion barrier of the patient is disrupted or removed by a skin resurfacing procedure to include, but not limited to, chemical peel, laser, mechanical abrasion, needling or rolling, and (iii) the cosmetic preparation from (i) is applied to the resurfaced skin of the patient during the healing phase.

Embodiments of the present invention include a cosmetic composition comprising somatic stem cells (also called adult stem cells) and growth factors. The somatic stem cells and growth factors are preferably isolated from autologous fat and/or bone marrow. The somatic stem cells may include, but are not limited to hematopoietic stem cells and mesenchymal stem cells. The somatic stem cells and growth factors, whether adipose derived or marrow derived, may be separated primarily by way of centrifugation. No further enrichment by plating or cell sorting is needed.

To harvest the mesenchymal stem cells and adipose growth factors, both direct lipectomy and suction assisted lipectomy may be performed on the patient. The suction lipectomy can be performed with or without tumescent and with or without adjuvant technologies like ultrasound assisted, laser liposuction, water-assisted, etc. The harvested fat is minced if direct lipectomy is performed. The fat is then separated by specific gravity using centrifugation, and the infranatant and cell pellet extracted from the tubes. To harvest the marrow-derived stem cells growth factors, needle aspiration or direct excision of marrow is performed and the effluent separated by specific gravity using centrifugation.

In some embodiments, the patient's fat is liposuctioned, and the lipoaspirate processed via centrifugation to isolate the stromal vascular fraction and growth factors, or peripheral blood is centrifuged to obtain a gradient containing an (a) upper layer of platelet poor plasma, (b) a middle layer containing platelet rich plasma, buffy coat and the top layer of red blood cells, and (c) a low layer of red blood cells and particulate. In some embodiments, layer (b) the middle layer containing platelet rich plasma, buffy coat and the top layer of red blood cells is used as the platelet rich plasma and growth factors.

Centrifugation may be carried out from about 1 to about 20 minutes depending on the gravitation force, which is preferably chosen from about 100 g to about 1000 g. The extract from centrifugation and isolation containing the somatic stem cells and growth factors may be used directly as a component of the cosmetic composition.

The form of the cosmetic composition may include, but is not limited to, a serum, cream, lotion, emollient or another composition for topical administration containing in part somatic stem cells and growth factors isolated from autologous fat or marrow. Preferably, the cosmetic composition serves two purposes: (i) to hydrate the skin creating a moist environment for wound healing and (ii) to keep the stem cells alive over a period of five to seven days. To these ends, some embodiments of the cosmetic composition may included one or more amino acids. The amino acid may include, but are not limited to, one or more or a combination of phenylalanine, valine, threonine, tryptophan, methionine, leucine, isoleucine, lysine, histidine, cystine, tyrosine, and glutamine. Further, some embodiments of the cosmetic composition may include a sugar alcohol. The sugar alcohol may include, but is not limited to glucose, fructose or other similar sugar alcohol. Optionally, other additives such as an anitboiotic and pyruvate may be included in the cosmetic preparation either individually or together. If the cosmetic composition is in the form of a cream, emollient or lotion then an oil or water base moisturizer may be included in the composition.

In some embodiments, the extract from centrifugation and isolation containing the somatic stem cells and growth factors may be used directly to form a serum that may be used as the cosmetic composition.

The somatic stem cells and growth factors are isolated from the intended recipient. Preferably, the somatic stem cells and growth factors are freshly isolated and used to form the cosmetic composition without significant delay—in some embodiments, within 48 hours, preferably within 5 hours, most preferably within 1 hour upon isolation of the somatic stem cells and growth factors.

Advantageously, the isolated cells and growth factors may be used directly to prepare the cosmetic composition. No further purification, cell culture, enrichment or by plating is needed. The lack of these steps not only simplifies the isolation, but also avoids contamination and preserves the stem cell's properties. No further cell culture or amplification is needed, as the amount of stem cells obtained by this method is sufficient. As the cosmetic composition comprising the somatic stem cells and growth factors is applied to the same individual from whom it was isolated, and does not contain any harmful by-products, it is very well tolerated.

Further embodiments of the invention include methods that comprise preparing the skin for the application of the cosmetic composition. Certain embodiments include the disrupting or removing the diffusion barrier of the stratum corneum that inhibits or prevents delivery of cells and growth factors larger then 600 Daltons into the dermis. Corneocytes, lamellar bodies, and, corneodesmosomes are specifically designed to prevent the loss of water and deter absorption of harmful environmental toxins. Substances that are hydrophilic or larger than 600 Daltons do not readily penetrate into the deeper dermis of the skin. For this reason, most cosmeceuticals are filled with proteins, acids and peptides with weights less than 600 Daltons. Cells, collagen and large proteins cannot diffuse through the outer layer of skin.

In some embodiments, a skin resurfacing step is utilized to remove or disrupt the epidermal diffusion barrier with or without removing the upper dermis to a degree sufficient for the somatic stem cells and growth factors of the cosmetic composition to effectively reach the dermis. The skin resurfacing step may include, but is not limited to, a chemical peel, laser treatment, mechanical abrasion, needling and rolling method, or other technique that provides removal of or disruption of the corneocytes and corneodesmosomes of the epidermis.

In some embodiments, methods of the present invention may accelerate and improve the aesthetic results after a skin resurfacing procedure. The wanted results from skin resurfacing—removal of heterochromias, pore size reduction, collagen and elastin production and wrinkle reduction—depend primarily on the speed and efficacy of the healing wound. The cosmetic preparation described above is used for post-procedure application to aid in the healing of the procedure to provide improved healing time and the final results.

Preferably, the cosmetic preparation comprising the somatic stem cells and growth factors is applied topically to the skin after skin resurfacing and continuously applied during the heeling process after the procedure. In some embodiments treatment time may range from about 5 to about 10 days.

Advantageously, the cell and growth factor harvest and resurfacing steps can be performed in less than 3 hours, preferably within 90 minutes or even 60 minutes, making it possible to apply the cell and growth factor preparation in the immediate setting after resurfacing.

Accordingly a method of treatment wherein somatic stem cells and growth factors are freshly isolated from an individual and reapplied to the skin of the same individual without significant delay, meaning within the same sitting or less than 3 hours, preferably within 90 minutes, most preferably within 60 minutes, is also part of the invention. This method advantageously makes a treatment with stem cells in or even outside a hospital possible.

Although the focus of this invention is the use of somatic stem cells for cosmetic purposes, a further aspect of the invention is the use of somatic stem cells and growth factors isolated from adipose or marrow for the treatment of skin conditions to include, but not limited to, scars, dermatitis, rosacea, psoriasis, etc. The efficacy is performed both by the stem cells themselves and via paracrine signaling to recipient site cells. As already stated the term stem cell according to the invention comprises not only living cells but also components thereof.

Embodiments of the invention further include to the use of somatic stem cells and/or growth factors for cosmetic purposes, preferably for application to ameliorate the appearance of the skin, e.g. by improving the skin texture, in particular for the application to aged skin, in particular to wrinkled and/or dimpled (cellulite) skin. The cells and growth factors are intended for serial, topical application to the skin over a 5 to 10 day period while healing from a resurfacing procedure.

Certain embodiments of the invention are illustrated by the following examples:

### Example 1:

This example describes the isolation of mesenchymal stem cells and growth factors from human adipose tissue and their cosmetic application after a skin resurfacing (LASER) procedure.

First, the skin upon the abdomen is cleaned with an antiseptic (ChloraPrep). Then a local anesthetic (e.g. 1% lidocaine with epinephrine) is injected subcutaneously with a standard needle (e.g. 27 gauge, ½″). Thereafter, a small incision is made and a standard liposuction infusion cannula is introduced into the subcutaneous fat. Injection of a tumescent solution (Klein's formula or a modification of) is performed. Once the appropriate analgesia and epinephrine effect have occurred, gentle suction-assisted lipectomy is performed with a suction cannula. The fat is harvested into sterile syringes and capped to facilitate transfer for centrifugation. The small nick incision is closed with tissue glue or a stitch.

The harvested fat is transferred to 15 mL conical tubes or capped and left in the original suction syringe. Each specimen is centrifuged for 10 min at 300 g to create a density gradient with the stromal vascular fraction (“SVF”) and liquid serum with growth factors at the bottom. The lysed cholesterol and supranatant cells are removed and, the SVF is re-suspended in the serum with growth factors. This is repeated for each tube of fat harvested.

The resultant solution is added to a serum bottle and mixed with an additive of choice. The additive preferably contains a natural antibiotic and sugar source for optimal cell survival. Additionally, the resultant serum can be added to a cream, lotion, or emollient.

A skin resurfacing procedure prepares the surface of the skin in the area where the stem cells and growth factors shall be applied. The face is gently de-greased and cleansed, and a physician or trained esthetician then performs an ablative LASER treatment. The operator is sure to reach pinpoint bleeding so as to be sure to remove the diffusion barrier of the epidermis.

Once complete, the stem cells and growth factor preparation produced as described above is applied to the injured skin. An additional emollient or lotion is applied overtop as needed. As the cosmetic preparation is applied to skin of the same person, from whom it is isolated, and does not contain any harmful by-products it is well tolerated. As the stem cells and growth factors are prepared very fast they can be applied to the person within less than an hour, even within 30 minutes, upon isolation (in the same sitting) making an ambulant treatment possible.

The patient continues to apply the stem cells and growth factor cosmetic to the healing skin for a period of 5 to 10 days. Application generally ceases once all skin has re-epithelialized.

### Example 2:

This example describes the isolation of hematopoietic stem cells and growth factors from human bone marrow and their cosmetic application after a skin resurfacing (chemical peel) procedure.

A standard bone marrow harvest is performed under general or local anesthesia. The area over the bone is prepped and draped in sterile fashion (in this instance over the iliac bone). Multiple percutaneous punctures are made with a large bore needle through the skin, subcutaneous tissue, and cortical bone to enter the bone marrow cavity. Marrow and blood are aspirated and stored in syringes.

The harvested marrow is transferred to 15 mL conical tubes or capped and left in the original suction syringe. Each specimen is processed by double centrifugation or by an established commercial separation system to isolate the hematopoietic stem cells, platelets and growth factors.

A skin resurfacing procedure prepares the surface of the skin in the area where the stem cells and/or growth factors shall be applied. The face is gently de-greased and cleansed, and a physician or trained esthetician then performs an ablative chemical peel procedure. The operator is sure to reach at least a light frost to ensure destruction of the epidermal diffusion barrier.

Once complete, the hematopoietic stem cells and growth factor preparation produced as described above is applied to the injured skin in the form of a serum or mixed into a base to produce a cream, lotion or emollient. As the cosmetic preparation is applied to skin of the same person, from whom it is isolated, and does not contain any harmful by-products it is well tolerated. As the stem cells and growth factors are prepared very fast they can be applied to the person within less than an hour, even within 30 minutes, upon isolation (in the same sitting) making an ambulant treatment possible.

The patient continues to apply the stem cells and growth factor cosmetic to the healing skin for a period of 5 to 10 days. Application generally ceases once all skin has re-epithelialized.

Having described certain embodiments of the present invention in detail above, one skilled in the art will understand aspects of the invention have broad application and is limited only by the following claims.

What is claimed is:

1. A cosmetic composition comprising somatic stem cells and growth factors derived from bone marrow or adipose tissue.

2. The cosmetic composition of claim 1, wherein the stem cells and growth factors are included in a serum, lotion, emollient, or cream.

3. The cosmetic composition of claim 1, wherein the composition further comprises at least one amino acid selected from the group consisting of phenylalanine, valine, threonine, tryptophan, methionine, leucine, isoleucine, lysine, histidine, cystine, tyrosine, and glutamine.

4. A method for treating skin of a patient comprising the steps of:
isolating somatic stem cells and growth factors from the patient;
preparing a cosmetic composition comprising the somatic stem cells and growth factors;
disrupting the epidermis diffusion barrier of the skin to be treated to form a disrupted epidermis diffusion barrier skin region; and
applying the cosmetic composition to the disrupted epidermis diffusion barrier skin region.

5. The method of claim 4 wherein the step of isolating the somatic stem cells and growth factors is performed using bone marrow or adipose tissue.

6. The method of claim 4 wherein the step of disrupting the epidermis diffusion barrier of the skin is selected from the group consisting of chemical peel, laser, mechanical abrasion, needling and rolling.

7. The method of claim 4, wherein the step of applying the cosmetic composition to a patient is performed within 48 hours after the step of preparing the cosmetic composition.

8. The method of claim 4, wherein the step of applying the cosmetic composition to a patient is performed within 5 hours after the step of preparing the cosmetic composition.

9. The method of claim 4, wherein the cosmetic composition is prepared from the patient's lipoaspirate or blood.
